(19) United States
(12) Patent Application Publication
(10) Pub. No.: US 2025/0090148 A1
(43) Pub. Date: Mar. 20, 2025

(54) MEDICAL DEVICES FOR TISSUE COLLECTION AND RELATED METHODS

(71) Applicant: Boston Scientific Medical Device Limited, Galway (IE)

(72) Inventors: Deepak Kumar SHARMA, Muzaffarnagar (IN); Hitendra PUROHIT, Vadodara (IN)

(73) Assignee: Boston Scientific Medical Device Limited, Galway (IE)

(21) Appl. No.: 18/887,428

(22) Filed: Sep. 17, 2024

Related U.S. Application Data
(60) Provisional application No. 63/583,641, filed on Sep. 19, 2023.

(51) Int. Cl. A61B 10/04; A61B 10/02; A61B 17/00; A61B 17/32; A61M 25/00

(52) U.S. Cl. CPC A61B 10/04; A61B 10/0266; A61B 17/320016; A61B 2010/0208; A61B 2017/00358; A61B 2017/320064; A61M 2025/0004; A61M 25/0074; A61M 2025/0095; A61M 25/0097

(57) ABSTRACT

A medical device may comprise a sheath and an expandable member. The expandable member may comprise a support member and a liner coupled to the support member. The liner may include a plurality of teeth. Proximal movement of the sheath may be configured to uncover the expandable member so that the expandable member transitions from a constrained configuration to a constrained configuration.

### CROSS-REFERENCE TO RELATED APPLICATIONS

This application claims the benefit of priority to U.S. Provisional Application No. 63/583,641, filed on Sep. 19, 2023, which is incorporated by reference herein in its entirety.

### TECHNICAL FIELD

The disclosure relates generally to medical devices and related methods. More particularly, the disclosure relates to medical devices used, for example, for tissue collection during biopsy or other procedures, and methods for using the devices.

### BACKGROUND

During medical procedures, such as endoscopic procedures, tissue may be acquired from a subject's body, such as from a wall of a body lumen of the subject. These tissue samples are often biopsied to determine the presence of a pathological disorder. Endoscopic biopsy forceps may be used in conjunction with an endoscope for taking tissue samples from the subject's body lumen for analysis. However, often, the samples must be obtained from deep within the body at locations that are difficult to access using standard forceps jaws (e.g., tissue from areas accessible only via tortuous biliary paths). In some cases, the quality or quantity of tissue accessible by a physician using standard forceps may not be sufficient for accurate diagnosis. Furthermore, forceps jaws may sometimes be difficult to maneuver as needed to obtain tangential bites or perpendicular bites. The devices and methods described herein may alleviate these deficiencies and one or more other deficiencies in the art. The scope of the current disclosure, however, is defined by the attached claims, and not by the ability to solve any specific problem.

### SUMMARY

Aspects of the disclosure relate to, among other things, a tissue collection device (e.g., for biopsy applications) and related methods. These aspects may include one or more of the features described below.

In an example, a medical device may comprise: a sheath; and an expandable member. The expandable member may comprise a support member; and a liner coupled to the support member. The liner may include a plurality of teeth. Proximal movement of the sheath may be configured to uncover the expandable member so that the expandable member transitions from a constrained configuration to an expanded configuration.

Any of the medical devices disclosed herein may have any of the following features, alone or in any combination. The support member may include a lattice. The support member may include a plurality of struts. The liner may include a first, proximal row of teeth and a second, distal row of teeth. In the constrained configuration, the second, distal row of teeth may form an approximately planar surface. Distal movement of the sheath may be configured to transition the expandable member from the expanded configuration to the constrained configuration. As the expandable member transitions from the expanded configuration to the constrained configuration, the first row of teeth may close before the second row of teeth close. The teeth may face at least partially in a proximal direction. A proximal end of the sheath may include a grip protruding radially outward from the proximal end of outer sheath. The grip may be configured to be manipulated by a user to move the sheath proximally or distally. In the expanded configuration, a distal portion of the expandable member may have a larger width than a proximal portion of the expandable member. In the expanded configuration, the expandable member may have a curved cone shape or a cylindrical shape. The medical device may include an anchoring assembly including an anchor. The anchor may be corkscrew-shaped. The medical device may further comprise a handle. The handle may include a first actuator configured to move the anchor proximally and distally. The handle may include a second actuator configured to rotate the anchor. The medical device may include a snare loop coupled to a distal end of the expandable member.

In another example, a medical device may comprise an expandable member having a support member; and a liner coupled to the support member. The liner may include a plurality of teeth. The medical device may further comprise an anchoring assembly, which may include an anchor and a control wire.

Any of the devices disclosed herein may include any of the following features in any combination. The medical device may further comprise a handle. The handle may include a first actuator coupled to the control wire and configured to move the anchor proximally or distally. The handle may include a second actuator coupled to the control wire and configured to rotate the anchor.

A medical method may comprise: moving a sheath proximally in order to expand an expandable member; extending an anchor distally to contact a tissue; moving the anchor proximally so that the tissue is within the expandable member; and moving the sheath distally to contract the expandable member, such that teeth of the expandable member secure the tissue to the expandable member.

Any of the methods disclosed herein may include any of the following steps, alone or in any combination. The method may include, after extending the anchor distally, rotating the anchor to secure the tissue to the anchor.

### BRIEF DESCRIPTION OF THE DRAWINGS

The accompanying drawings, which are incorporated in and constitute a part of this specification, illustrate exemplary embodiments that, together with the written description, serve to explain the principles of this disclosure.

FIGS. 1A and 1B illustrate an exemplary tissue collection device of the current disclosure;

FIGS. 2A-2B, FIGS. 3A-3E, and FIGS. 4A-4C depict aspects of a distal end of the tissue collection device of FIG. 1;

FIG. 5 depicts the distal end of FIGS. 3A-4C along with a tissue anchor;

FIGS. 6A-6C illustrate aspects of a handle of the tissue collection device of FIG. 1.

FIGS. 7A-7G depict steps of a medical method performed with the tissue collection device of FIG. 1.

FIGS. 8A-8C depict an alternative distal end for use with the tissue collection device of FIG. 1.

FIG. 9 illustrates an alternative distal end of the tissue collection device of FIG. 1.

### DETAILED DESCRIPTION

The disclosure is now described with reference to an exemplary tissue collection device that may be used in an endoscopic procedure, such as a biopsy. However, it should be noted that reference to this particular procedure is provided only for convenience and not intended to limit the disclosure. A person of ordinary skill in the art would recognize that the concepts underlying the disclosed device and application method may be utilized in any suitable procedure, medical or otherwise. The disclosure may be understood with reference to the following description and the appended drawings, wherein like elements are referred to with the same reference numerals, where feasible.

Exemplary embodiments of the disclosure describe a medical device that may be advanced into a body lumen through an introduction device (e.g., through a working channel of an endoscope, duodenoscope, or other type of scope device, including but not limited to colonoscopes, endoscopic ultrasound (EUS) scopes, cystoscopes, ureteroscopes, bronchoscopes, catheters, and the like). Exemplary embodiments of the medical device include an expandable member having teeth on an inner surface thereof. An anchor (e.g., a hook) may extend through a center of the expandable member. The disclosed medical devices may increase the volume of tissue that may be obtained, as compared with conventional devices. The disclosed medical devices may increase a hit rate of a procedure. More conclusive tissue samples may be obtained, with a shorter procedure time. Embodiments of the disclosed device may also increase the maneuverability of the device through a tortuous lumen of a subject's body. Furthermore, the disclosed devices may help to obtain tissue in a single pass, rather than requiring repeated tissue engagement. This may decrease damage or trauma to surrounding tissues due to multiple insertions of alternative medical devices. Furthermore, the disclosed devices may result in a shorter procedure. The devices disclosed herein may also have increased cost-effectiveness and ease of manufacturability as compared to biopsy forceps, due to a use of fewer components.

It may be understood that both the foregoing general description and the following detailed description are exemplary and explanatory only and are not restrictive of the invention, as claimed. As used herein, the terms “comprises,” “comprising,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “diameter” may refer to a width where an element is not circular. The term “exemplary” is used in the sense of “example,” rather than “ideal.” For ease of description, portions/regions/ends of the device and/or its components are referred to as proximal and distal ends/regions. It should be noted that the term “proximal” is intended to refer to ends/regions closer to a user of the device, and the term “distal” is used herein to refer to ends/regions further away from the user. Similarly, saying that a component extends “distally” indicates that a component extends in a distal direction (away from a user), and saying that a component extends “proximally” indicates that a component extends in a proximal direction (toward a user). Further, as used herein, the terms “about,” “approximately” and “substantially” indicate a range of values within +/−10% of a stated or implied value. Additionally, terms that indicate the geometric shape of a component/surface refer only to approximate shapes.

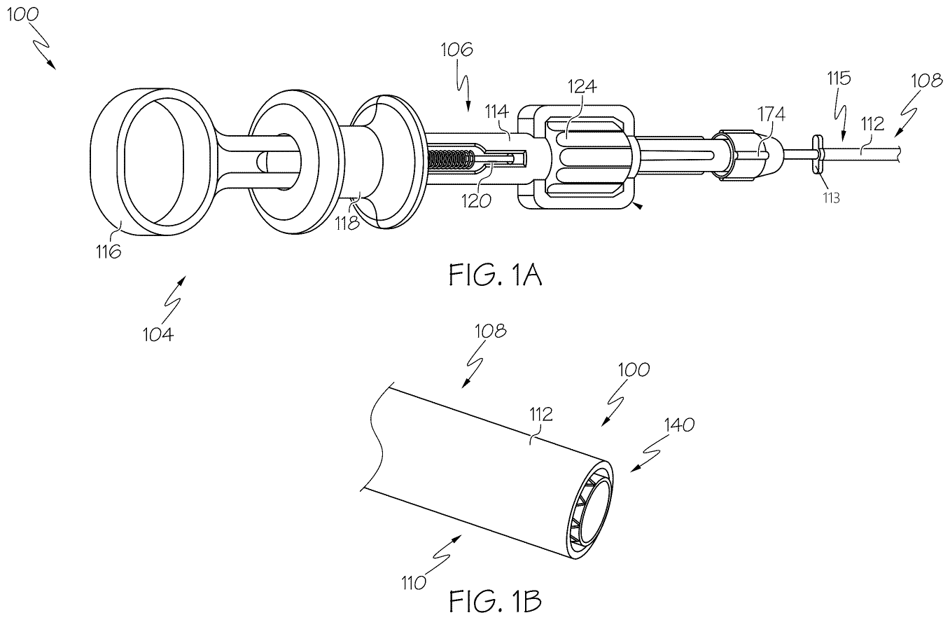

FIGS. 1A and 1B illustrate an exemplary embodiment of a tissue collection medical device (“medical device”) 100. FIG. 1A shows details of a proximal portion of medical device 100, and FIG. 1B shows details of a distal portion of medical device 100. As shown in FIG. 1A, medical device 100 may include a proximal assembly 104, including a handle 106. Medical device 100 may include a shaft 108, for example, extending from a distal end of handle 106. Medical device 100 may also include a distal assembly 110 at a distal end of shaft 108. As discussed in detail below, movement of various portions of handle 106 may allow for steering, articulating, and/or manipulating the components of distal assembly 110.

Handle 106 may include a body 114. A proximal end of body 114 may include a ring 116 (e.g., a thumb ring). Handle 106 may also include a plurality of actuators. For example, handle 106 may include a first movable member/actuator, which may be a spool 118. Handle 106 may further include a second movable member/actuator, which may be a knob 124. As discussed in detail below, spool 118 and knob 124 may be used to actuate elements of distal assembly 110.

Shaft 108 may include an outer sheath 112, having any suitable material, such as a stainless steel coil, polytetrafluoroethylene (PTFE), braided polymer, or a Nitinol (nickel tin) tube (e.g., a hypotube). A proximal end of sheath 112 may include an actuator, which may be a grip 113. Grip 113 may protrude radially outward from a proximal end of sheath 112. As discussed below, grip 113 may be contacted and moved proximally or distally by a user in order to move sheath 112 proximally and/or distally.

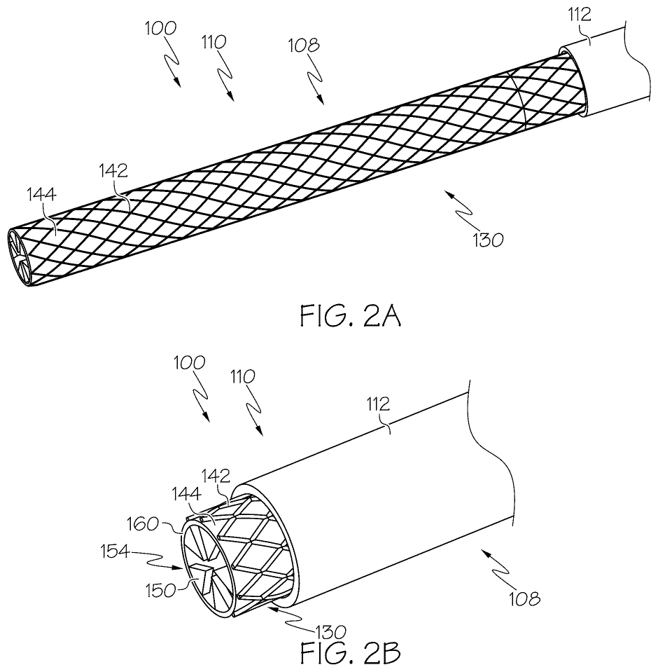

FIG. 2A shows a partial tearaway view of distal assembly 110 in a first, constrained/contracted configuration. In the constrained configuration, outer sheath 112 may extend to a distalmost end of shaft 108, as shown in FIG. 1B. The constrained configuration of FIG. 2A may be utilized while navigating shaft 108 to a procedure site (e.g., via a working channel of an endoscope) or when retracting shaft 108 from a procedure site.

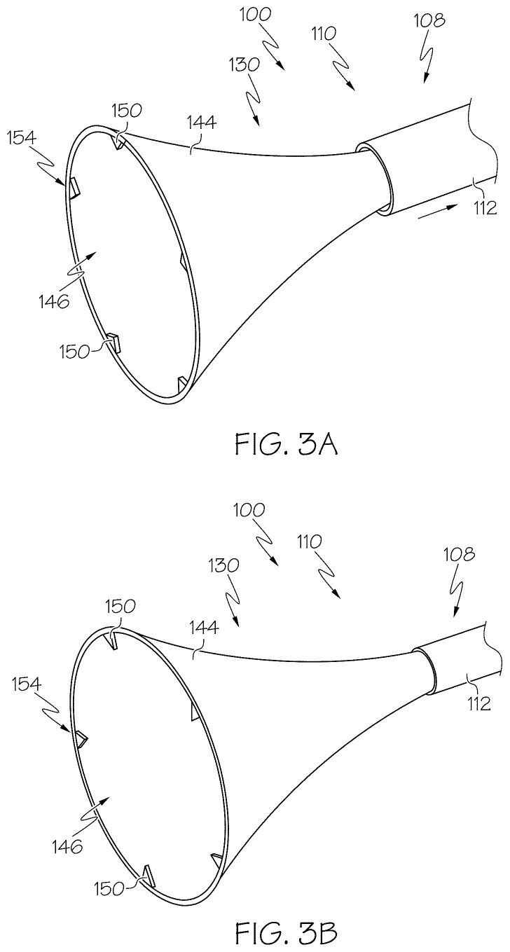

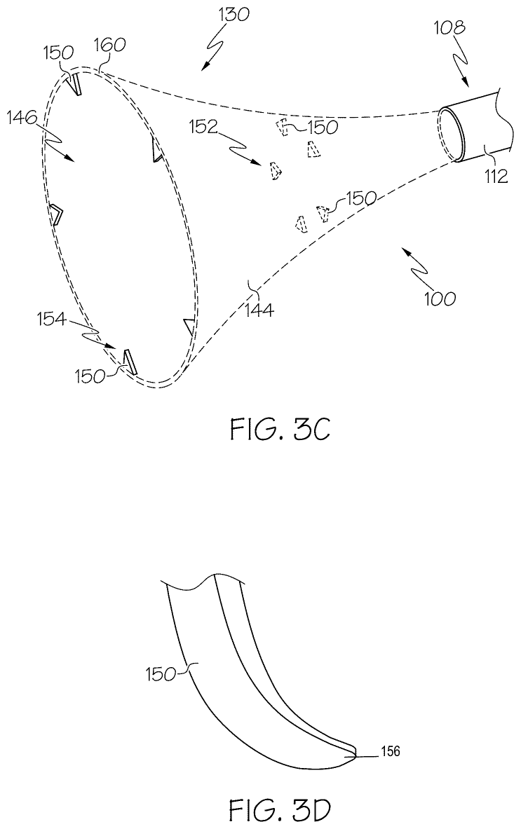

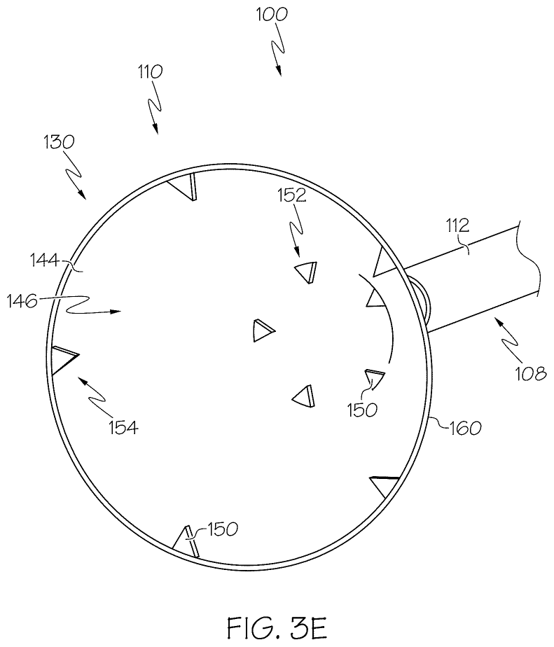

As shown in FIG. 2A, an inner member 140 may extend within outer sheath 112. A distal end of inner member 140 may include an expandable member 130. In some examples, inner member 140 may extend proximally to a proximal end of shaft 108. In other examples, inner member 140 may be disposed only at a distal portion of shaft 108. Expandable member 130 may include a lattice 142 and a liner 144. Lattice 142 may be a support member that supports and gives a desired shape to liner 144. Lattice 142 may include wires of shape-memory material, such as Nitinol (nickel titanium) or another suitable material. Lattice 142 may have any suitable pattern, such as a braided, crisscrossing wires, a woven pattern, a knit pattern, etc. As discussed in further detail below, lattice 142 may be biased to an expanded configuration (FIG. 3B). For example, lattice 142 may be biased by heat setting. Outer sheath 112 may maintain lattice 142 in the constrained configuration of FIG. 2A. Outer sheath 112 may be movable in a proximal direction in order to transition expandable member 130 to the expanded configuration. FIG. 2B shows outer sheath 112 partially retracted but without sufficient retraction for expandable member 130 to begin transitioning to the expanded configuration.

Liner 144 may be coupled (e.g., fixedly coupled) to lattice 142. In some examples, as shown, liner 144 may be coupled to an inner surface of lattice 142, such that lattice 142 is on an outer surface of liner 144. Alternatively, liner 144 may be coupled to an outer surface of lattice 142, such that lattice 142 is coupled to an inner surface of lattice 142. Liner 144 may be constructed from PTFE or any other suitable material. In some examples, liner 144 may be elastic, such that it may stretch to transition from the contracted configuration of FIGS. 2A and 2B to the expanded configuration of FIG. 3B. In other examples, liner 144 may be inelastic and may include folds in the contracted configuration of FIGS. 2A and 2B. Liner 144 may be sufficiently thin that, as lattice 142 automatically expands to the biased, expanded configuration (FIG. 3B), liner 144 also transitions to the expanded configuration. Liner 144 may also be sufficiently thin that it is able to be compressed within outer sheath 212. Liner 144 may be sufficiently thick or otherwise resilient that it is able to engage tissue, as discussed below.

In some examples, liner 144 may be omitted. In other examples, a portion of liner 144 may be omitted, and portions of lattice 142 may include liner 144. A mesh structure of lattice 142 may allow for fluids (e.g., blood, bile, etc.) to pass through lattice 142 without liner 144. Lattice 142 may remove a need to have separate fenestrations formed in medical device 100.

FIGS. 3A-3E shows features of expandable member 130 in various stages of transitioning to the biased, expanded configuration. FIGS. 3A-3E omit lattice 142 for ease of illustration. It will be appreciated that lattice 142 may positioned on an outside surface of liner 144. Alternatively, liner 144 may have shape memory properties such that liner 144 is biased to the expanded configuration, and lattice 142 may be omitted.

In FIG. 3A, outer sheath 212 may be partially retracted in a proximal direction, as shown by the arrow. A distal portion of expandable member 130, including liner 144, may begin to expand radially outward. The configuration of FIG. 3A may be a partially open (or partially closed) configuration.

In FIG. 3B, outer sheath 112 has been further retracted proximally, such that expandable member 130 is in a fully expanded configuration. Expandable member 130 may define a cavity 146. Cavity 146 may have an open distal end. In some examples, an inner surface of liner 144 may define a surface of cavity 146. In the expanded configuration of FIG. 3B, expandable member 130 may have a trumpet shape. In other words, liner 144 and/or lattice 142 may taper radially outward in a distal direction, such that expandable member 130 has a curved cone shape. An outer surface of liner 144 and/or lattice 142 may be concave, such that expandable member 130 flares outward. A proximal portion of expandable member 130 may have a smaller diameter/width than a distal portion of expandable member 130, in the expanded configuration. In other words, the distal portion of expandable member 130 may have a larger diameter/width than the proximal portion of expandable member 130. Alternatively, expandable member 130 may have other shapes, such as approximately hemispherical, bell, straight cone, cylindrical, or bulbous shapes.

An inner surface of liner 144 may have a plurality of teeth 150, which may extend radially inward into cavity 146. FIG. 3C shows liner 144 as being transparent to reveal details of teeth 150, with expandable member 130 in the expanded configuration. FIG. 3E shows a partially proximally-facing view of expandable member 130, which shows teeth 150. As shown in FIGS. 3C and 3E, teeth 150 may be arranged in a first, proximal row 152 and a second, distal row 154. Although two rows 152, 154 are depicted in the Figures and described below, it will be appreciated that expandable member 130 may include any suitable number of rows of teeth. First row 152 and second row 154 may have any suitable number of teeth. For example, as shown in FIGS. 3C and 3E, each of first row 152 and second row 154 may include five teeth. However, such a number is merely exemplary. The two rows 152, 154 of teeth 150 may help with a hit rate of procedures using medical device 100.

First row 152 of teeth 150 may extend around a circumference of liner 144 at a first axial/longitudinal location of liner 144, such that first row 152 forms a ring of teeth 150. A first plane that is perpendicular to a longitudinal axis of expandable member 130 may extend through all of teeth 150 of first row 152. Second row 154 of teeth 150 may extend around a circumference of liner 144 at a second axial/longitudinal location of liner 144, such that second row 154 forms a ring of teeth 150. A second plane that is perpendicular to a longitudinal axis of expandable member 130 may extend through all of teeth 150 of second 154. For example, as shown in the Figures, second row 154 may extend around a distal rim 160 of liner 144.

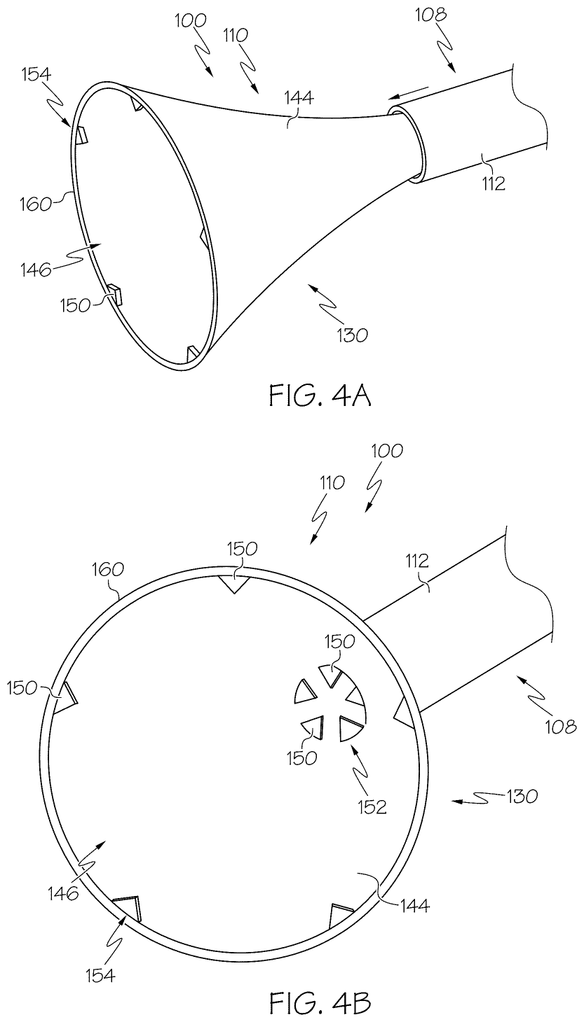

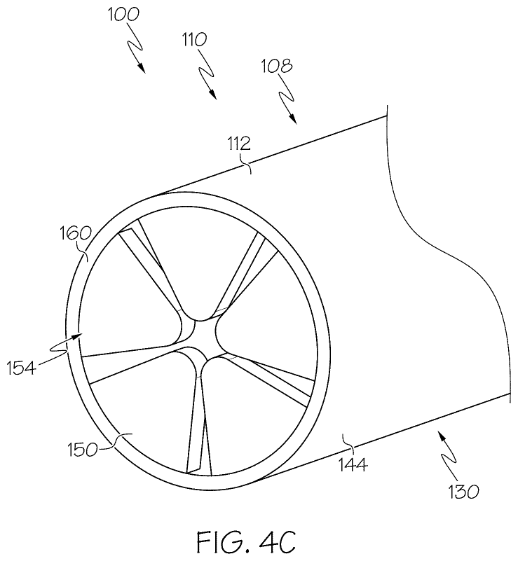

In the expanded configuration (FIGS. 3B, 3C, and 3E), teeth 150 in first row 152 and second row 154 may be spaced apart from other teeth in the same row 152, 154. In the contracted configuration (see FIG. 2B), teeth 150 of second row 154 may be closer to one another. For example, teeth 150 may contact adjacent teeth 150, and tips of teeth 150 may meet or may nearly meet at a center of expandable member 130 (e.g., near a central longitudinal axis of shaft 108). Thus, teeth 150 of second row 154 may form an approximately closed distal end in the contracted configuration. Although not shown in FIG. 2B, teeth 150 of first row 152 may have a similar configuration in the contracted configuration, as discussed above with respect to FIG. 4B.

Teeth 150 may be evenly spaced around an inner surface of liner 144. Evenly spaced teeth 150 may prevent localized pressure points that could lead to tissue damage when teeth 150 engage tissue, as disclosed below. Teeth 150 of first, proximal row 152 may be closer together than teeth 150 of second, distal row 154 because of a relatively smaller diameter/width of expandable member 130 at a location of first, proximal row 152. Teeth 150 may have any suitable shape. For example, as shown in FIG. 3D, teeth 150 may have a curved, sickle shape. A point 156 of teeth 150 may face proximally, to increase a grip on tissue. Additionally or alternatively, point 156 may face radially inward, toward a central longitudinal axis of expandable member 130. Teeth 150 may have a radius of curvature that may help to reduce tissue trauma. The shape of FIG. 3D is merely exemplary, and teeth 150 may have any suitable shape. Each of teeth 150 may have the same shape or may have differing shapes (e.g., straight, triangle, serrated, barbed, or other shapes). Teeth 150 may have any suitable size (e.g., approximately 0.3 mm to approximately 1.0 mm), for example, depending on how many of teeth 150 are used.

FIG. 3D shows an exemplary shape of teeth 150. The shape of the teeth may be curved or sickle-shaped. The curve may face proximally and or radially inward. The curvature of teeth 150 may help to envelop the tissue, providing a more secure grasp on the target tissue. The sickle shape of the teeth 150 may make controlled cuts and minimize damage to surrounding healthy tissue. The teeth radius size may be between 0.3 mm to 1 mm to assist in precisely targeting smaller tissue areas and reducing tissue trauma while allowing an enveloping action.

FIGS. 4A-4C show expandable member 430 transitioning from the expanded configuration to the constrained configuration. In FIGS. 4A and 4B, the outer sheath 112 is moved distally to transition expandable member 430 to a partially contracted configuration. Sheath 112 may constrain proximal portions of expandable member 430. The configuration of FIGS. 4A and 4B may be similar to the partially expanded configuration shown in FIG. 3A. FIG. 4B shows a somewhat proximally facing view, to show details of teeth 150. As shown in FIG. 4B, as expandable member 130 begins to contract, first row 152 of teeth 150 may grow closer to one another, as outer sheath 112 nears an axial position of first row 152. As discussed below, as teeth 150 of first row 152 move closer to one another (i.e., close), the teeth 150 of first row 152 may grip into tissue and may assist with retaining tissue within expandable member 130. As sheath 112 continues to move distally, teeth 150 of first row 152 may transition to a fully closed configuration (similar to the configuration of second row 154 shown in FIG. 4C, discussed below). For example, first row 152 of teeth 150 may form an approximately planar shape, with or without gaps between teeth 150. Teeth 150 of first row 152 may remain in a partially open configuration when tissue is received within expandable member 130, to accommodate the tissue.

FIG. 4C shows expandable member 130 in a fully constrained/collapsed configuration. The configuration is similar to that of the configuration shown in FIG. 2A. To transition expandable member 130 from the partially expanded configuration of FIGS. 4A-4B to the fully constrained/collapsed configuration of FIG. 4C, outer sheath 112 may be moved farther proximally, to a position near a distalmost end of expandable member 130 (e.g., near rim 160). Teeth 150 of second row 154 may move together (close), as described above. In some examples, teeth 150 of second row 154 may form an approximately planar wall that can retain tissue within cavity 146.

As discussed in further detail below, teeth 150 may be configured to grip and/or sever tissue that is received within expandable member 130. Because first row 152 and second row 154 of teeth 150 have an approximately planar arrangement in the retracted configuration and grab tissue about a central point (e.g., along a central longitudinal axis of expandable member 130), expandable member 130 may produce fewer tissue crush artifacts in a tissue sample, as compared with biopsy forceps. Two rows 152, 154 of teeth 150 may have improved anchoring and retention of tissue as compared to a single row of teeth or an arrangement of teeth of a biopsy forceps.

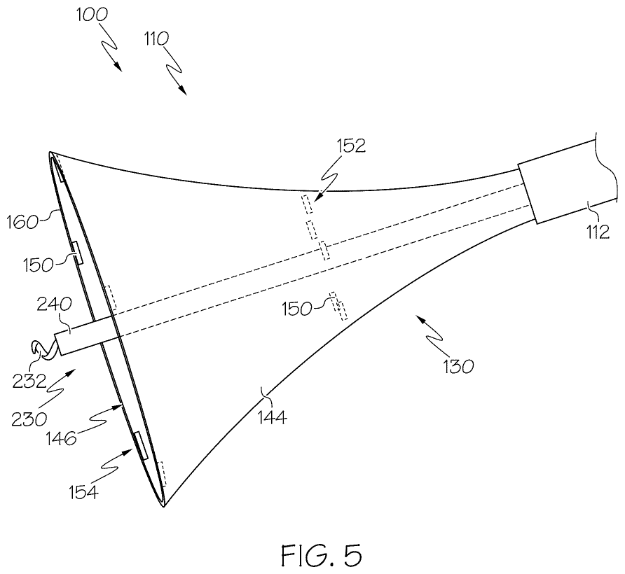

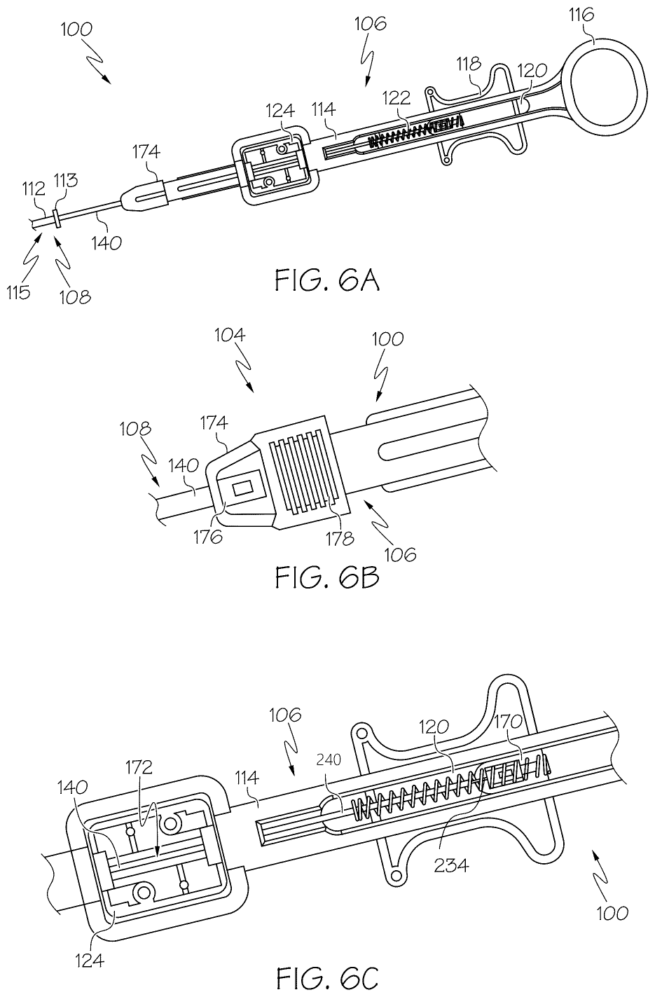

FIG. 5 shows an anchoring assembly 230 of medical device 100. Anchoring assembly 230 may help with anchoring tissue and gaining more depth and volume of tissue in a single pass. Anchoring assembly 230 may include an anchor 232 coupled to a control wire 234 (FIGS. 6A and 6C). In some examples, anchor 232 may include a metal wire that may be formed integrally with control wire 234 or formed separately from control wire 234 and coupled thereto. Control wire 234 (or another control member, such as a cable) may extend through an inner sheath 240. Inner sheath 240 may extend through a lumen of inner member 140 (and, thus, outer sheath 112) and through cavity 146 of expandable member 130. Inner sheath 240 may be a tubular structure that defines a lumen. Control wire 234 may extend through the lumen of inner sheath 240 (e.g., a sleeve). In some examples, inner sheath 240 may include a coil or a hypotube of any suitable material (e.g., PTFE). As discussed below, at least portions of inner sheath 240 may have a square or other non-round cross-section.

Anchor 232 may be configured to be moved distally (extended) and/or proximally (retracted) with respect to inner member 140 via distal and/or proximal movement of control wire 234. In examples, as shown in FIG. 5, anchor 232 may move proximally and distally along with inner sheath 240. In other examples, anchor 232 may be movable with respect to inner sheath 240. Anchor 232 may include a helix, a hook, a barb, a lance, trocar, or other structure. Anchor 232 may be formed from metal. In the example shown in FIG. 5, anchor 232 may be helical shaped (e.g., corkscrew shaped) and may have a pointed distal tip. As discussed in further detail below, anchor 232 may be used to grip tissue and to position tissue relative to expandable member 130 so that the tissue may be captured within expandable member 130. In some examples, anchor 232 may be rotated into tissue to secure anchor 232 to the tissue. Anchor 232 may be the same material or different from the remainder of the control wire 234.

FIGS. 6A-6C show details of proximal assembly 104. FIGS. 6A and 6C are cross-sectional views, and FIG. 6B shows portions of proximal assembly 104 as being transparent to reveal details of proximal assembly 104. As discussed above, proximal assembly 104 may include a handle 106 having one or more actuators for manipulating aspects of distal assembly 110, as described below.

Spool 118 of handle 106 may be sized and shaped to be grasped by a user. Body 114 of handle 106 may include a slot 120, which may extend longitudinally through a portion of body 114. A distal end of slot 120 may be spaced distally from ring 116. A portion of spool 118 may extend into a portion of slot 120, such that spool 118 is movable (e.g., longitudinally movable distally and/or proximally) along slot 120. Slot 120 may define a range of movement for spool 118. As discussed in detail below, spool 118 may be movable within slot 120, for example, proximally and/or distally, to control one or more aspects of the medical device. Additionally, handle 106 may include a spring or other type of biasing element 122, which may bias spool 118 to a proximal or distal position.

Spool 118 may include an internal projection 170 (labeled in FIG. 6C), for example, that extends within a portion of slot 120. Internal projection 170 or another portion of spool 118 may be coupled to a proximal end of control wire 234, for example, via a crimp or other suitable structure (e.g., a ferrule) that is fixed to control wire 234. A proximal end of biasing element 122 may be coupled to a proximal end of projection 170. Alternatively, biasing element 122 may not be coupled to projection 170, but may abut or otherwise interact with projection 170 as spool 118 is advanced distally within slot 120. Biasing element 122 may bias spool 118 proximally. For example, biasing element 122 may compress as spool 118 is moved distally, and biasing element 122 may urge spool 118 proximally (e.g., to the position shown in FIG. 6A) once the distal force on spool 118 is removed.

As described in further detail below, movement of spool 118 may control the actuation of distal assembly 110. For example, longitudinal movement of spool 118 in a first direction (e.g., in the distal direction) may actuate control wire 234 to move in a distal direction and advance anchor 232 distally relative to inner member 140 and expandable member 130. Movement of the spool 118 in the distal direction may advance anchor 232 until it contacts tissue of a subject's body. Longitudinal movement of spool 118 in a second, opposite direction (e.g., in the proximal direction) may retract control wire 234 and anchor 232 relative to inner member 140 and expandable member 130. For example, control wire 234 and anchor 232 may be moved proximally such that they are covered by inner member 140 and outer sheath 112. As discussed below, movement of the spool 118 in the proximal direction may allow for tissue T anchored to anchor 232 to be pulled into the expandable member 130.

Knob 124 may be disposed on a distal portion of body 114 of handle 106. As discussed in detail below, knob 124 may be rotatable, for example, clockwise and/or counterclockwise, to control one or more aspects of distal assembly 110, as discussed below. For example, knob 124 may be rotatable about a longitudinal axis (e.g., a central longitudinal axis) of medical device 100.

As particularly shown in FIG. 6C, a proximal portion of control wire 234 may be fixedly coupled to knob 124. A coupler, for example, inner sheath 240, may be attached or otherwise positioned around a proximal portion of control wire or around an entire length of control wire 234. A portion of inner sheath 240 within knob 124 (or an entire length of inner sheath 240) may include one or more flat outer surfaces. For example, a portion or an entirety of inner sheath 240 may include a square cross-section with four flat outer surfaces. In some aspects, inner sheath 240 may extend proximally to spool 118, for example, through biasing element 122 and/or to internal projection 170. In other examples, inner sheath 240 may terminate within or just proximally of knob 124. Knob 124 may surround control wire 234 and inner sheath 240, such that knob 124 engages with inner sheath 240 to help rotate inner sheath 240 and control wire 234 as knob 124 rotates. For example, knob 124 may define a lumen 172 that has a keyed shape with inner sheath 240 (e.g., lumen 172 may have a square cross-section). Control wire 234 and/or inner sheath 240 may be movable proximally and/or distally with respect to knob 124 and may be rotatably fixed with respect to knob 124.

In these aspects, rotation of knob 124 (e.g., clockwise or counterclockwise) may also rotate control wire 234, (e.g., clockwise or counterclockwise about central to a longitudinal axis of control wire 234 and/or a central longitudinal axis of shaft 108). For example, the user may rotate knob 124 (e.g., about a central longitudinal axis of knob 124) to rotate control wire 234 (via inner sheath 240). Rotation of control wire 234 may rotate anchor 232, such that anchor 232 engages with and secures the tissue. For example, rotation of knob 124 may cause a corkscrew-shaped anchor 232 to twist into tissue to secure anchor 232 to the tissue.

A distalmost portion of handle 106 may include a coupling portion 174 that couples a proximal end of inner member 140 to handle 106. Coupling portion 174 is depicted as being transparent in FIG. 6B in order to show details of coupling portion 174. A proximal end of inner member 140 may include a crimp sleeve 176, a bearing, a ferrule, or another structure for coupling inner member 140 to coupling portion 174. Coupling portion 174 may include threads 178 for coupling the coupling portion 174 to body 114 of handle 106. As coupling portion 174 is screwed onto (or otherwise coupled to) handle 106, coupling portion 174 may engage with crimp sleeve 176 to couple inner member 140 to coupling portion 174.

As shown in FIGS. 1 and 6A, proximal assembly 104 may also include a proximal portion of outer sheath 112. As mentioned above, a proximal end 115 of outer sheath 112 may include grip 113. Grip 113 protrudes radially outward from proximal end 115 of outer sheath 112. Grip 113 may be sized and shaped so as to be contacted by a hand of a user. Grip 113 may be manipulated by a user to move outer sheath 112 proximally and/or distally. As discussed above, proximal movement of outer sheath 112 may result in transition of expandable member 130 into the expanded configuration of expandable member 130 (shown in FIGS. 3B, 3C, 3E, and 5). Distal movement of outer sheath 112 may progressively cover expandable member 130, transitioning it to the constrained/collapsed configuration (shown in FIGS. 1, 2A, 2B, and 4C). In alternatives, handle 106 may include one or more other actuators to control extension (distal movement) and retraction (proximal movement) of outer sheath 112.

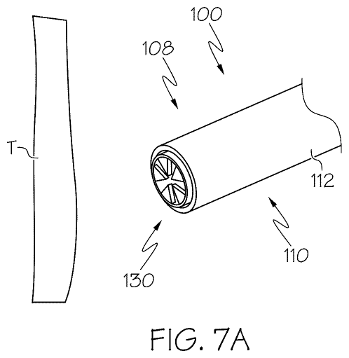

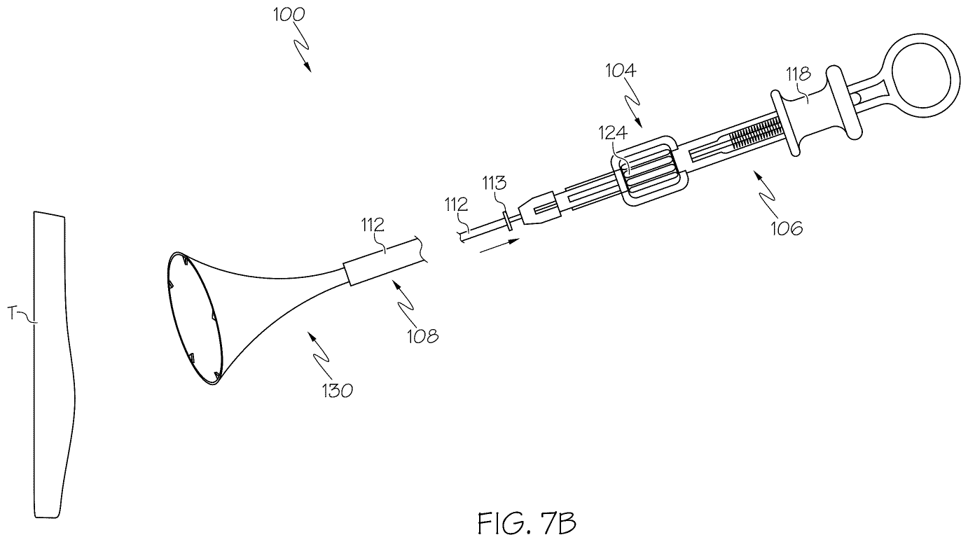

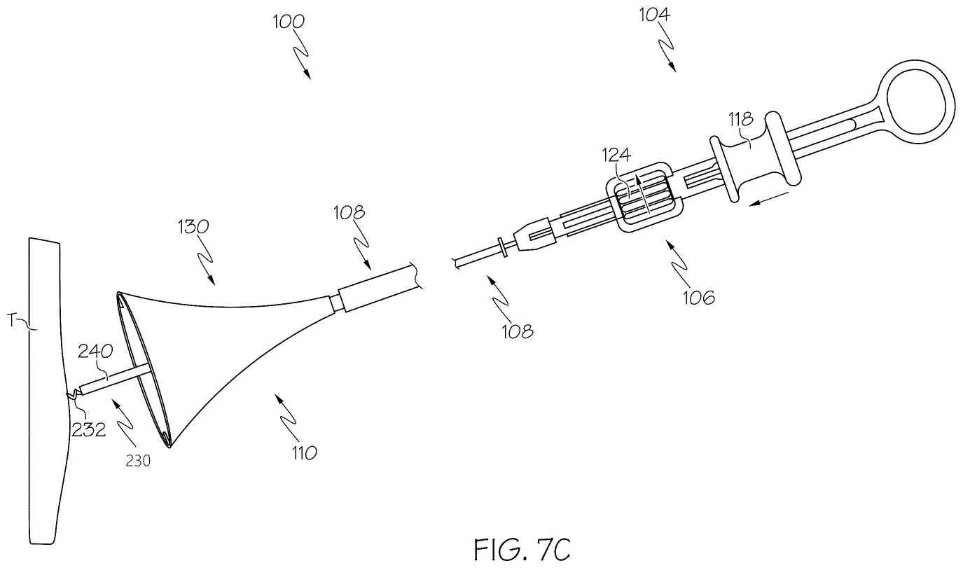

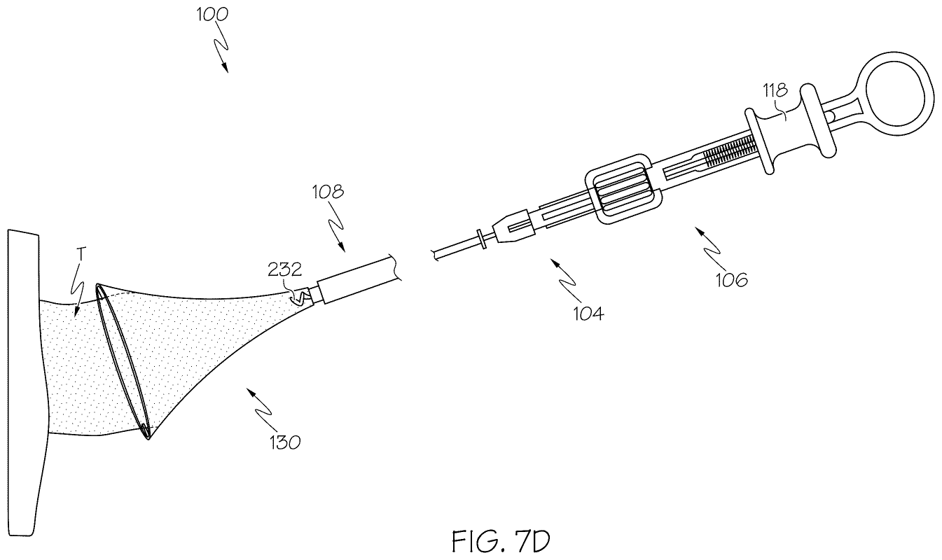

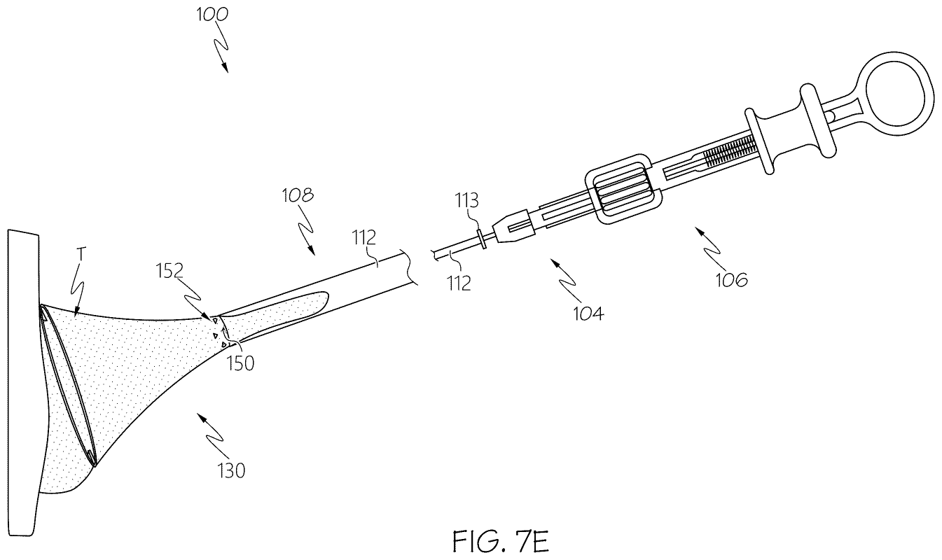

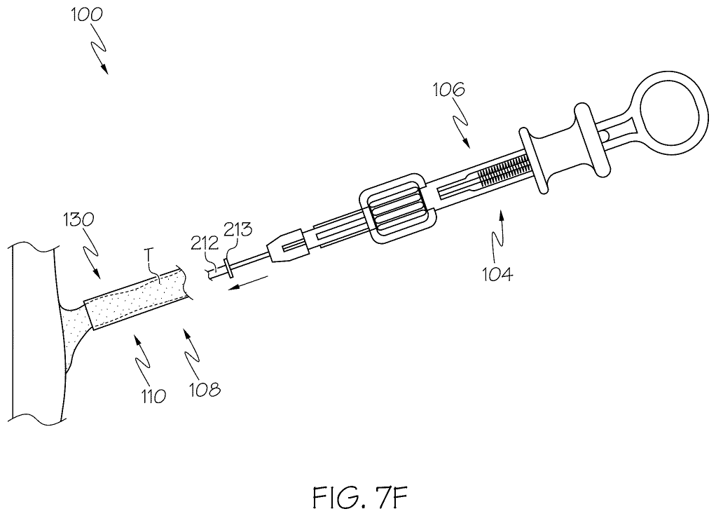

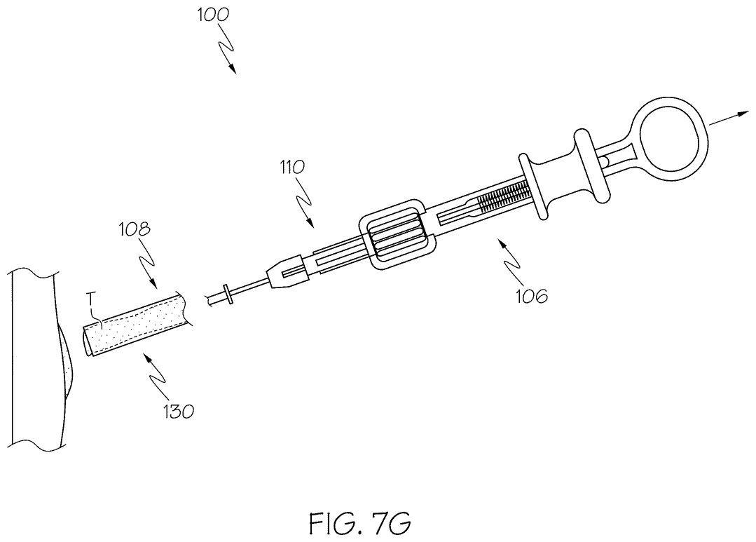

FIG. 7A-7F depicts a method of using medical device 100 to collect/acquire a tissue sample. Although FIGS. 7A-7F and the description below depict and describe a particular order of method steps, it will be appreciated that some or all of the steps below may be repeated or may be performed in alternative orders. In some of FIGS. 7A-7E, liner 144 is depicted as being transparent for ease of illustration.

FIG. 7A shows distal assembly 110 of medical device 100 with expandable member 130 in a constrained/contracted configuration. Outer sheath 112 may extend to a distal end (e.g., a distalmost end) of expandable member 130, constraining expandable member 130 within outer sheath 112. Anchor 232 (not shown in FIG. 7A) may also be retracted proximally within outer sheath 112 and expandable member 130. In some examples, anchor 232 may be proximal of the proximalmost end of expandable member 130 in the configuration of FIG. 7A.

An introduction device (e.g., any type of scope device) may be introduced into the body of a subject (e.g., through a body orifice) and positioned such that it is suitably positioned in a body lumen. Shaft 108 of medical device 100 may then inserted be inserted into the body lumen via the introduction device. Alternatively, shaft 108 may be inserted into the body of a subject without using an introducing device. During insertion of medical device 100, distal assembly 110 may be in a constrained configuration, described above with respect to FIGS. 1, 2A, 2B, and 4C.

As shown in FIG. 7B, an expandable member 130 may then be transitioned into the expanded configuration. To transition the expandable member 130 into the expanded configuration, the user contact grip 113 of outer sheath 112 in order to move outer sheath 112 proximally with respect to expandable member 130, as shown by the arrow in FIG. 7B. In the alternate, outer sheath 112 may be retracted by manipulating other controls on the handle 106 of the medical device.

As the outer sheath 112 moves proximally, it may progressively uncover expandable member 130. Expandable member 130 may transition to a biased, expanded configuration, as described above with respect to FIGS. 3A-3E. The user may optionally cease expansion of expandable member 130 at a partially expanded configuration, as shown in FIG. 3A. For example, the user may desire for expandable member 130 to have a smaller size than the fully expanded configuration.

As shown in FIG. 7C, a user may then move spool 118 distally (as shown by the arrow) to move anchor 232 distally toward tissue T. Anchor 232 may be extended so that it is distal to a distalmost end of expandable member 130. Thus, anchor 232 may be used to engage with tissue T without requiring expandable member 130 to be pushed against tissue T. Knob 124 may be rotated, as shown by the arrow to rotate anchor 232 so that it engages with and secures/anchors into tissue T. For example, a corkscrew-shaped anchor 232 may be screwed into tissue T.

Then, as shown in FIG. 7D, a user may move spool 118 proximally (as shown by the arrow) in order to move anchor 232 proximally relative to expandable member 130. For example, anchor 232 may retract within expandable member 130. As discussed with respect to FIG. 7C, anchor 232 may have been secured to tissue T, such that, as anchor 232 is moved proximally, tissue T is pulled into expandable member 130.

Thereafter, as shown In FIG. 7E, the operator may move outer sheath 112 distally using grip 113, as shown by the arrow. As discussed above with respect to FIGS. 4A and 4B, a proximal portion of expandable member 130 may begin to contract. First row 152 of teeth 150 may engage with tissue T that is drawn into expandable member 130, as described above. First row 152 of teeth 150 may grip/anchor onto tissue T within expandable member 130, thereby securing the tissue to expandable member 130. As shown in FIG. 7E, tissue T may extend proximally of first row 152 of teeth 150, within expandable member 130. The proximal portions of tissue T may be within a collapsed portion of expandable member 130.

As shown in FIG. 7F, the user may continue to move grip 113 distally (as shown by the arrow) to move outer sheath 112 distally over the expandable member 130. The outer sheath 112 may move in a distal direction until it fully covers expandable member 130. Second row 154 of teeth 150 (not shown in FIG. 7F) may close, as described above with respect to FIG. 4C in order to retain tissue T within expandable member 130. Second row 154 of teeth 150 may provide further grip/anchoring on tissue T and may provide a distal, closed end of expandable member 130.

As shown in FIG. 7F, a user may move an entirety of handle 106 proximally (shown by the arrow) in order to withdraw the collected tissue T from the body. Handle 106 may be withdrawn with a jerk to get tissue purchase. In some examples, second row 154 of teeth 150 may help with severing tissue T. Handle 106 may be moved further proximally to retract shaft 108, holding collected tissue T, from the body lumen (e.g., through a working channel of an introduction device).

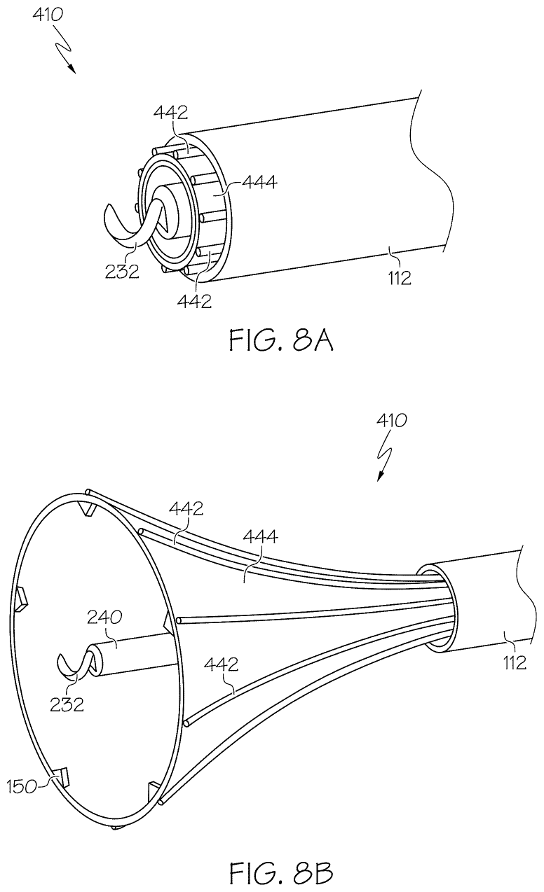

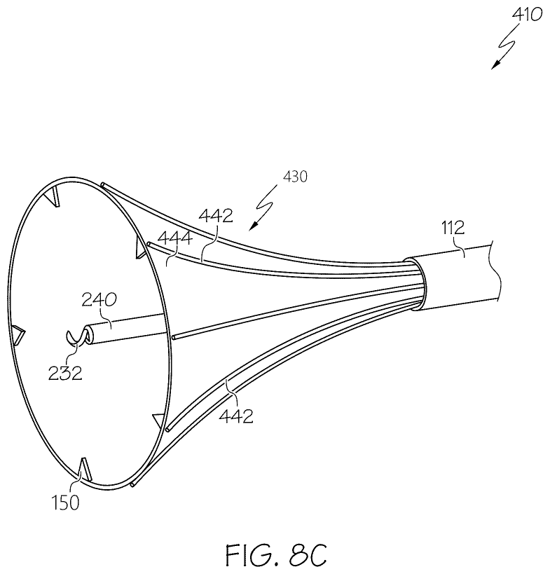

FIGS. 8A-8C depict an alternative distal assembly 410. Unless otherwise specified herein, distal assembly 410 may have any of the features of distal assembly 110 and may be used with any of the methods described herein. Distal assembly 410 may be used in conjunction with proximal assembly 104, described above. FIG. 8A shows an expandable member 430 of distal assembly 410 in a contracted configuration. FIG. 8B shows expandable member 430 in a partially expanded configuration. FIG. 8C shows the expandable member 430 in a fully expanded configuration.

Whereas expandable member 130 includes a lattice 142, expandable member 430 of distal assembly 410 may include a plurality of struts 442. Struts 442 may be support members that provide a shape to liner 444 and give structure to liner 444. In some examples, a plurality of struts 442 may be understood to constitute a single support member. Struts 442 may include a plurality of longitudinally extending members (e.g., wires, cables, etc.). Struts 442 may be coupled to an outer surface of a liner 444, having any of the properties of liner 144 described above. In the contracted configuration (FIG. 8A), struts 442 may be approximately parallel to one another and to a central longitudinal axis of expandable member 430. In the expanded configurations (FIGS. 8B and 8C), struts 442 may flare outward in a distal direction, radially outward from the central longitudinal axis of expandable member 430. Struts 442 may be curved and may bend radially outward. Struts 442 may be evenly spaced around an outer surface of liner 444. Any suitable number of struts 442 may be utilized, to provide a desired amount of support to liner 444.

Similarly to lattice 142, struts 442 may have shape memory properties (e.g., may be made from Nitinol and heat set into an expanded configuration or otherwise have a biased, expanded configuration). Struts 442 may be biased into the expanded configuration of FIG. 8C.

In some examples, expandable member 430 may lack a liner, and struts 442 alone may act as expandable member 430. Struts 442 may have one or more teeth integrated therein (e.g., embedded).

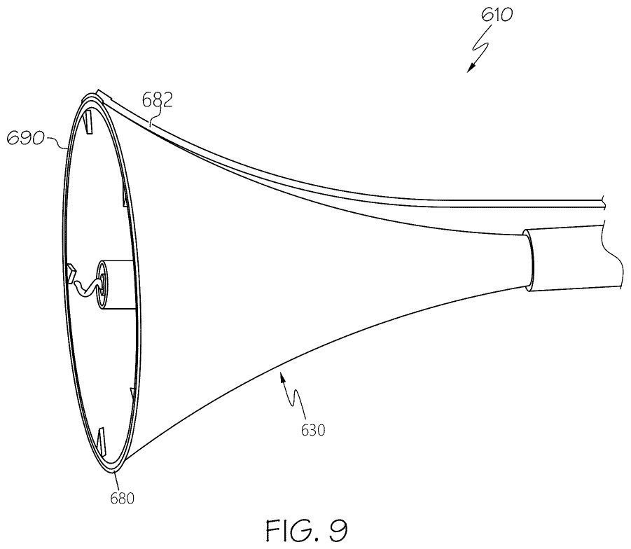

FIG. 9 depicts another alternative distal assembly 610. Distal assembly 610 may have any of the properties of distal assemblies 10, 410 unless otherwise specified herein, and may be used with any of the methods described herein. Distal assembly 610 may be used in conjunction with proximal assembly 104.

The distal assembly 610 may include an expandable member 630. Expandable member 630 may have any properties of expandable members 130 and 430, described above. The expandable member 630 may include a snare loop 680 attached to a distal end (e.g., a distalmost rim 690) of expandable member 630. Snare loop 680 may have any of the features of any snare loop known in the art and may be biased to an expanded configuration (shown in FIG. 9). Snare loop 680 may be fixed to expandable member 630 via any suitable means. For example, mechanical fasteners such as screws, rivets, loops, or clips or adhesive may be used to attach snare loop 680 to expandable member 630. In some examples, snare loop 680 may be secured to rim 690 (e.g., by loops along rim 690), such that snare loop 680 may move relative to rim 690.

A shaft 682 may extend proximally from snare loop 680. A control wire (not shown) or other control element (e.g., cable, thread, etc.) may be received within a lumen of shaft 682 and operatively coupled to snare loop. Proximal retraction of the control wire may cause snare loop 680 to transition to a closed configuration (not shown), thereby collapsing snare loop 680. Distal movement of the control wire may cause snare loop 680 to transition to an open configuration (shown in FIG. 9), thereby expanding snare loop 680. The control wire 234 may be actuated by an element of handle 106 or by a separate handle or actuator. Alternatively, the snare loop 680 may be controlled passively, such that snare loop 680 may contract as expandable member 630 contracts and expand as expandable member 630 expands.

Distal assembly 610 may be particularly useful for cancer resection. Snare loop 680 may be actuated to resect tissue (similarly to other snare loops), and expandable member 630 may retain the resected tissue. Distal assembly 610 may help to present seeding of cancerous cells because the tissue is retained within expandable member 630.

Although biopsy and tissue resection are referenced above, it will be appreciated that the devices disclosed herein may be utilized in other procedures, such as removing or crushing stones (e.g., biliary stones) or retrieving foreign objects. Furthermore, the devices disclosed herein may be utilized for procedures such as mechanical thrombectomy. In alternatives, suction may be utilized to draw tissue into the expandable members described herein. In such examples, anchoring assembly 230 may be omitted.

While principles of this disclosure are described herein with the reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

We claim:

1. A medical device, comprising:
a sheath; and
an expandable member, comprising:
a support member; and
a liner coupled to the support member, wherein the liner includes a plurality of teeth;
wherein proximal movement of the sheath is configured to uncover the expandable member so that the expandable member transitions from a constrained configuration to an expanded configuration.

2. The medical device of claim 1, wherein the support member includes a lattice.

3. The medical device of claim 1, wherein the support member includes a plurality of struts.

4. The medical device of claim 1, wherein the liner includes a first, proximal row of teeth and a second, distal row of teeth.

5. The medical device of claim 4, wherein, in the constrained configuration, the second, distal row of teeth form an approximately planar surface.

6. The medical device of claim 4, wherein distal movement of the sheath is configured to transition the expandable member from the expanded configuration to the constrained configuration, and wherein, as the expandable member transitions from the expanded configuration to the constrained configuration, the first row of teeth close before the second row of teeth close.

7. The medical device of claim 4, wherein the teeth face at least partially in a proximal direction.

8. The medical device of claim 1, wherein a proximal end of the sheath includes a grip protruding radially outward from the proximal end of outer sheath, wherein the grip is configured to be manipulated by a user to move the sheath proximally or distally.

9. The medical device of claim 1, wherein, in the expanded configuration, a distal portion of the expandable member has a larger width than a proximal portion of the expandable member.

10. The medical device of claim 1, wherein, in the expanded configuration the expandable member has a curved cone shape or a cylindrical shape.

11. The medical device of claim 1, further comprising an anchoring assembly including an anchor.

12. The medical device of claim 11, wherein the anchor is corkscrew-shaped.

13. The medical device of claim 11, further comprising a handle, wherein the handle includes a first actuator configured to move the anchor proximally and distally.

14. The medical device of claim 13, wherein the handle includes a second actuator configured to rotate the anchor.

15. The medical device of claim 1, further comprising a snare loop coupled to a distal end of the expandable member.

16. A medical device, comprising:
an expandable member, comprising:
a support member; and
a liner coupled to the support member, wherein the liner includes a plurality of teeth; and
an anchoring assembly including an anchor and a control wire.

17. The medical device of claim 16, further comprising a handle, wherein the handle includes a first actuator coupled to the control wire and configured to move the anchor proximally or distally.

18. The medical device of claim 17, wherein the handle includes a second actuator coupled to the control wire and configured to rotate the anchor.

19. A medical method comprising:
moving a sheath proximally in order to expand an expandable member;
extending an anchor distally to contact a tissue;
moving the anchor proximally so that the tissue is within the expandable member; and
moving the sheath distally to contract the expandable member, such that teeth of the expandable member secure the tissue to the expandable member.

20. The medical method of claim 19, further comprising, after extending the anchor distally, rotating the anchor to secure the tissue to the anchor.
